(19) United States
(12) Patent Application Publication
(10) Pub. No.: US 2019/0209638 A1
(43) Pub. Date: Jul. 11, 2019

(54) NATURAL COMPOSITION HAVING ALPHA-GLUCOSIDASE ACTIVITY INHIBITION EFFECT AND USED FOR REGULATING THE ABSORPTION OF BLOOD GLUCOSE AND CARBOHYDRATE

(71) Applicant: Syi Biotechnology Co.,Ltd., Taichung City (TW)

(72) Inventors: Hsiu-Man Lien, Taichung City (TW); Chia-Chang Chen, Taichung City (TW); Sheau-Jiun Chang, Taichung City (TW); Chao-Lu Huang, Taichung City (TW); Jui-Ting Hsu, Taichung City (TW)

(21) Appl. No.: 15/863,351

(22) Filed: Jan. 5, 2018

(51) Int. Cl. A61K 36/54; A61K 36/734; A61P 3/10

(52) U.S. Cl. CPC A61K 36/54; A61P 3/10; A61K 36/734

(57) ABSTRACT

A natural composition that includes C. osmophloeum and C. pinnatifida. The C. osmophloeum is an aqueous extract of Cinnamomum osmophloeum leaves. The Crataegus pinnatifida is an aqueous extract of C. pinnatifida fruit. The weight ratio of the C. osmophloeum to the C. pinnatifida is 1:0.05-2, the preferred ratio is 1:0.5-1.5, and the optimal ratio is 1:0.9-1.1. The pharmaceutical composition has an α-glucosidase inhibition effect. The natural composition can further inhibit α-amylase to regulate the absorption of blood glucose and carbohydrate.

### BACKGROUND OF THE INVENTION

### 1. Field of the Invention

The present invention relates to a natural composition. In particular, the present invention relates to a natural composition having an α-glucosidase activity inhibition effect and the composition being used for regulating the absorption of blood glucose and carbohydrate.

### 2. Description of the Prior Art

Diabetes mellitus is a chronic hyperglycaemic metabolic disease, and is also one of the most serious chronic diseases. The main pathogenesis is that the insulin is secreted insufficiently or an organism tissue is unable to utilize glucose effectively, thereby resulting in excessive glucose content in the blood. It is known that the insulin is secreted by β cells in the pancreas, which has a blood glucose regulation effect and can stimulate fat and muscle cells to transport glucose. When a human body is short of insulin secretion or poor insulin sensitivity due to obesity, aging and other factors, the blood glucose concentration will increase and cause diabetes. Diabetic patients may have the symptoms such as thirst, polyuria, blurred vision and weight loss, and long-term hyperglycemia that may lead to dysfunction and failure of various organs.

Diabetes mellitus is mainly divided into type I diabetes mellitus, type II diabetes mellitus, gestational diabetes mellitus, and other types of diabetes mellitus (Diabetes Care. 33:S63-S65). Type I diabetes mellitus is required to be treated with insulin for the whole life of a patient because the pancreas is unable to secrete insulin normally. Type II diabetic patients account for about 90-95% of all diabetic patients. The main pathogenesis of Type II diabetes is the resistance of peripheral tissues to insulin, which leads to the decrease of insulin utilization rate or the relative deficiency of insulin, and thus leading to the rise of blood glucose (Clin. Invest. Med. 18:303-11).

At present, the drugs used to treat type II diabetic patients are mainly divided into four categories:

1. Sulfonyl urea compound for stimulating the pancreas to secrete insulin;

2. Biguanides for inhibiting a hepatic gluconeogenesis effect and improving the sensitivity of liver and skeletal muscle to insulin;

3. Thiazolidinedione for reducing fat decomposition and improving the sensitivity of liver and skeletal muscle to insulin; and

4. α-glucosidase inhibitor for reducing the absorption of carbohydrates in the intestine.

However, the first three types of drugs are prone to severe side effects such as hypoglycemia, lactic acidemia and the like. The α-glucosidase inhibitor has relatively minor side effects. With respect to the α-glucosidase inhibitor, the drug that is widely used at present is acarbose, wherein a pseudo-tetrasaccharide structure can inhibit both α-glucosidase and α-amylase at the same time to achieve the effect of delaying the rise of the blood glucose.

With the improvements of human living standards, diabetic patients are found all over the world. More than 90% of diabetic patients are diagnosed as type II diabetic patients. Diabetes mellitus is a chronic systemic metabolism-abnormal disease, and often leads to the host of many complications, such as heart disease, stroke, neurological problems, eye diseases and the like. Accordingly, the development of a drug or health care food having a blood glucose regulation effect and capable of delaying the occurrence of diabetes mellitus complications is very urgent.

Diabetes mellitus is known as “thirst quenching disease” in ancient Chinese books. For thousands of years, many Chinese herbs have been widely used to prevent or treat diabetes mellitus. Unlike modern western medicine, many Chinese herbal medicine compounds are used in the traditional Chinese medicine. For example, a plurality of single prescriptions (single Chinese herbs) are combined in the same drug formulation. The main purpose is to improve therapeutic effect or reduce the side effects and toxicity of a single prescription. Although the prior art has many references regarding the regulation of blood glucose with Chinese herbal medicine, most of the references merely discuss a single Chinese herbal medicine or an extract thereof. The studies of Chinese herbal medicine compounds are very rare. Most of the studies regarding the regulation of blood glucose with Chinese herbal medicine compounds are limited to traditional formulations. Therefore, designing a Chinese herbal medicine natural composition, particularly a Chinese herbal medicine natural composition having an α-glucosidase activity inhibition effect to be used for regulating the absorption of blood glucose and carbohydrate, is an important issue to be solved by the present invention.

### SUMMARY OF THE INVENTION

The present invention relates to a natural composition, and in particular to a pharmaceutical composition having an α-glucosidase activity inhibition effect.

One object of the present invention is to provide a natural composition having an α-glucosidase activity inhibition effect, wherein the natural composition comprises C. osmophloeum and C. pinnatifida.

To achieve the object above of the present invention, in the natural composition, the C. osmophloeum is an aqueous extract of C. osmophloeum leaves.

To achieve the object above of the present invention, in the natural composition, the C. pinnatifida is an aqueous extract of C. pinnatifida fruit.

To achieve the object above of the present invention, in the natural composition, the weight ratio of the C. osmophloeum to the C. pinnatifida is 1:0.05-2.

To achieve the object above of the present invention, in the natural composition, the preferred weight ratio of the C. osmophloeum to the C. pinnatifida is 1:0.5-1.5.

To achieve the object above of the present invention, in the natural composition, the optimal weight ratio of the C. osmophloeum to the C. pinnatifida is 1:0.9-1.1.

To achieve the object above of the present invention, in the natural composition, the natural composition can further comprise a carrier, an excipient, a diluent and an adjuvant which are pharmaceutically acceptable.

Another object of the present invention is to provide a use of the natural composition, wherein the drug can regulate blood glucose.

To achieve the object above of the present invention, in the use of the natural composition, the blood glucose is regulated by regulating the absorption of carbohydrate.

To achieve the object above of the present invention, in the use of the natural composition, wherein the absorption of carbohydrate is regulated by inhibiting the activity of α-glucosidase.

To achieve the object above of the present invention, in the use of the natural composition, the drug can further inhibit the activity of α-amylase.

To achieve the object above of the present invention, in the use of the natural composition, the drug is administrated by means of oral administration, intravenous injection, or injection.

To achieve the object above of the present invention, in the use of the natural composition, the drug is administrated in the form of capsule, pastille, powder or oral liquid.

To achieve the object above of the present invention, in the use of the natural composition, the daily dose of the natural composition is 0.05-128 g.

To achieve the object above of the present invention, in the use of the natural composition, the preferred daily dose of the natural composition is 0.275-32 g

To achieve the object above of the present invention, in the use of the natural composition, the optimal daily dose of the natural composition is 0.275-1.6 g.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention. FIG. 1 is the inhibition rate of natural aqueous extracts and acarbose for α-glucosidase at different concentrations, wherein the values are expressed as means±standard deviation of triplicate tests;

FIG. 2A is the inhibition rate of natural aqueous extracts for α-amylase at different concentrations, wherein the values are expressed as means±standard deviation of triplicate tests;

FIG. 2B is the inhibition rate of acarbose for α-amylase at different concentrations, wherein the values are expressed as means±standard deviation of triplicate tests;

FIG. 3A is the inhibition rate of natural aqueous extracts in different ratios for α-glucosidase at different concentrations, wherein the values are expressed as means±standard deviation of triplicate tests; and

FIG. 3B is the inhibition rate of natural aqueous extracts in different ratios for α-amylase at different concentrations, wherein the values are expressed as means±standard deviation of triplicate tests.

### DETAILED DESCRIPTION OF THE PREFERRED EMBODIMENT

Aspects of the invention are disclosed in the following description and related drawings directed to specific embodiments of the invention. Alternate embodiments may be devised without departing from the scope of the invention.

Sample extraction method: cleaning all the samples with deionized water, and drying the samples in an oven at 40-45° C.; adding 100 g dried product in 1 L deionized water, and performing extraction for 60 minutes in a reflux manner; filtering and quantitatively concentrating the liquid extract to 100 ml, freezing and drying respectively to be powders; respectively adding the extracted powders in quantitative deionized water to the concentration of 25.6 mg/ml, and mixing until the powder is dissolved completely. The liquid sample extract (natural aqueous extract) is taken as a test material to evaluate the inhibition effects for α-glucosidase and α-amylase activities.

Mixture preparation: cleaning Cinnamomum osmophloeum and Crataegus pinnatifida with deionized water, and performing extraction for 60 minutes in a reflux manner; filtering the liquid extract, freezing and drying respectively to be powders; dissolving the extract in deionized water to the concentration of 100 mg/ml, shaking io until the extract is dissolved completely; and forming the natural aqueous extract mixtures MIX-1-5 according to the volume percent of mixture compositions as shown in table 1. For example, forming MIX-1 with 40% by volume of natural aqueous extract of the C. osmophloeum and 60% by volume of natural aqueous extract of C. pinnatifida. The mixtures are used to evaluate the inhibition effects for α-glucosidase and α-amylase activities. In the present invention, the C. osmophloeum refers to the leaves of Cinnamomum osmophloeum; and the C. osmophloeum refers to the fruit of Crataegus pinnatifida.

TABLE 1
Compositions of mixtures.

| | Part | MIX-1 | MIX-2 | MIX-3 | MIX-4 | MIX-5 |
|---|---|---|---|---|---|---|
| Scientific name | used | Composition (%) | | | | |
| Cinnamomum | Dried | 40.0 | 50.0 | 66.7 | 85.7 | 92.3 |
| osmophloeum | leaves | | | | | |
| Crataegus | Dried | 60.0 | 50.0 | 33.3 | 14.3 | 7.7 |
| pinnatifida | ripe | | | | | |
| | fruit | | | | | |

### Evaluation of the Inhibition Effect for α-Glucosidase Activity

Respectively, adding 0.3 ml natural aqueous extract and acarbose (the serial diluted concentrations are respectively: 0.01, 0.02, 0.04, 0.08, 0.16, 0.32, 0.64, 1.28 and 2.56 mg/ml) in a 0.06 ml 0.2 U/ml α-glucosidase phosphoric acid buffer solution in pH 6.8; mixing and placing in a 37° C. thermostatic water bath to perform reaction for 10 minutes; respectively adding 0.06 ml 2.5 mM p-Nitrophenol α-D-Glucoside (p-NPG) phosphoric acid buffer solution in pH 6.8; mixing and placing in a 37° C. thermostatic water bath to perform reaction for 20 minutes; finally adding 0.24 ml 0.1M Na₂CO₃, and ending the reaction.

After the reaction is completed, the absorbance value of the product ρ-nitrophenol (ρ-NP) decomposed from the ρ-NPG in the mixture is measured by using an ELISA reader at the wavelength 405 nm, and then the inhibition percent for α-glucosidas activity is calculated according to the absorbance value. The calculation formula is as follows:

$$Enzyme activity inhibition rate ( \% ) = \frac{[ ( Ac - Acb ) - ( As - Ab ) ]}{Ac - Acb} \times 100 \% ;$$

where

Ac: control set (0.3 ml sterile water+0.06 ml 0.2U/ml α-glucosidase);

As: sample set (0.3 ml sample+0.06 ml 0.2U/ml α-glucosidase);

Acb: control blank set (0.3 ml sterile water+0.06 ml 0.1M phosphoric acid buffer solution); and

Ab: sample blank set (0.3 ml sample+0.06 ml 0.1M phosphoric acid buffer solution).

Evaluation on the Inhibition Effect for α-Amylase Activity

Respectively, adding 0.25 ml natural aqueous extracts (the serial diluted concentrations are respectively: 0.4, 0.8, 1.6, 3.2, 6.4, 12.8 and 25.6mg/ml) and acarbose (the serial diluted concentrations are respectively: 0.01, 0.02, 0.04, 0.08, 0.16, 0.32, 0.64 and 1.28 mg/ml) in a 0.25 ml 5U/ml α-amylase solution, mixing and placing at the room temperature 25° C. to perform reaction for 10 minutes; then respectively adding 0.25 ml 0.5% starch matrix solution, mixing and placing at the room temperature 25° C. to perform reaction for 10 minutes; finally adding 0.5 ml DNS reagent, performing color reaction for 5 minutes in a 95° C. thermostatic water bath; and quickly cooling the reactant in an ice water bath;

0.1 ml reactant above is added in 0.4 ml deionized water. The absorbance value of the reduced carbohydrate decomposed from the starch matrix solution is measured by using the ELISA reader at the wavelength 570 nm, and then the inhibition percent for α-amylase activity is calculated according to the absorbance value. The calculation formula is as follows:

$$Enzyme activity inhibition rate ( \% ) = \frac{[ ( Ac - Acb ) - ( As - Ab ) ]}{Ac - Acb} \times 100 \% ;$$

where

Ac: control set (0.25 ml sterile water+0.25 ml 5 U/ml α-amylase);

As: sample set (0.25 ml sample+0.25 ml 5 U/ml α-amylase);

Acb: control blank set (0.25 ml sterile water+0.25 ml 0.02M phosphoric acid buffer solution containing 6 mM NaCl); and

Ab: sample blank set (0.25 ml sample+0.25 ml 0.02M phosphoric acid buffer solution containing 6 mM NaCl).

### Embodiment 1: Evaluation on the Inhibition Effect of Natural Aqueous Extracts for α-glucosidase Activity

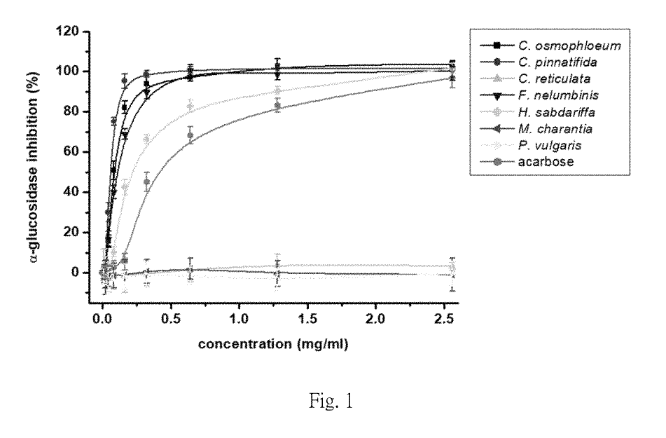

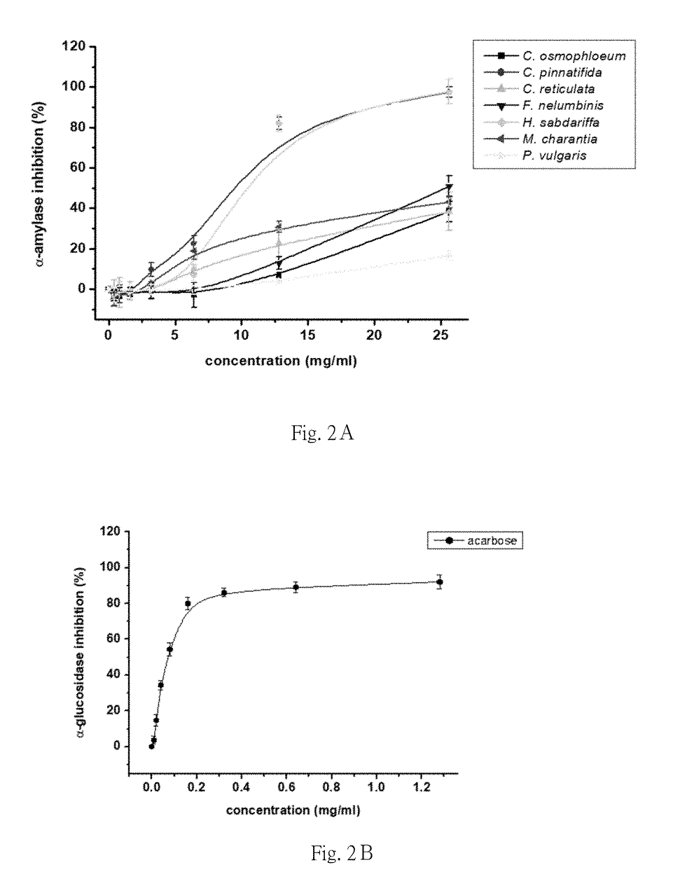

As shown in FIG. 1, the inhibition capabilities of different natural aqueous extracts and acarbose (the serial diluted concentrations are 0.01-2.56 mg/ml) for α-glucosidase activity are measured respectively, and the result shows that the inhibition effects of different natural aqueous extracts for α-glucosidase activity are correlated with the dose. At the concentration 0.32 mg/ml, the inhibition rates of the natural aqueous extracts (C. Osmophloeum, C. pinnatifida, F. nelumbinis and H. sabdariffa) for a-glucosidase activity are respectively 93.86%, 98.74%, 89.72% and 66.36% which are higher than the 45.08% enzyme activity inhibition rate of acarbose at the same concentration. However, the inhibition effects of other natural aqueous extracts, such as P. vulgaris, M. charantia and C. reticulate, for α-glucosidase activity are poor. As shown in FIG. 2, the 50% inhibitory concentrations (IC50) of C. osmophloeum, C. pinnatifida, F. nelumbinis, H. sabdariffa and acarbose for α-glucosidase activity are respectively 0.081±0.005, 0.055±0.001, 0.101±0.002, 0.22±0.017 and 0.422±0.053 mg/ml, the values are expressed as means±standard deviation of triplicate tests.

### Embodiment 2: Evaluation on the Inhibition Effect of Natural Aqueous Extracts for α-amylase Activity

The inhibition capabilities of different natural aqueous extracts (the concentrations are 0.4-25.6 mg/ml) and acarbose (the concentrations are 0.01-1.28 mg/ml) for α-amylase activity are measured respectively; the result in FIG. 2 shows that the inhibition effects of different natural aqueous extracts for α-amylase activity are correlated with the dose. The inhibition rate of different natural aqueous extracts at the concentration 6.4 mg/ml, such as C. pinnatifida, M. charantia and C. reticulate, for α-amylase activity are respectively 22.51, 18.83 and 9.92% (FIG. 2A); and the inhibition rate of acarbose at the concentration 0.04 mg/ml for α-amylase activity is 34.43% (FIG. 2B).

As shown in FIG. 2, the 50% inhibitory concentrations (IC50) of C. pinnatifida, F. nelumbinis, H. sabdariffa and acarbose for α-amylase activity are respectively 8.67±0.29, 25.23±1.92, 9.91±0.51 and 0.069±0.005 mg/ml, the values are expressed as means±standard deviation of triplicate tests.

TABLE 2
IC50 values of aqueous extracts from natural products
for α-amylase and α-glucosidase inhibition.

IC50 (mg/ml)
aqueous extract

| Analyte | α-glucosidase | α-amylase |
|---|---|---|
| C. osmophloeum | 0.081 ± 0.005 | 29.31 ± 1.145 |
| C. pinnatifida | 0.055 ± 0.001 | 8.670 ± 0.278 |
| C. reticulata | — | 37.93 ± 12.81 |
| F. nelumbinis | 0.101 ± 0.002 | 25.23 ± 1.793 |
| H. sabdariffa | 0.220 ± 0.017 | 9.906 ± 0.513 |
| M. charantia | — | 29.11 ± 3.466 |
| P. vulgaris | — | 49.41 ± 7.220 |
| acarbose | 0.422 ± 0.053 | 0.069 ± 0.005 |

The values are expressed as means ± standard deviation of triplicate tests.
— means no experiment

### Embodiment 3: the Influence of Natural Aqueous Extracts in Different Ratios for α-glucosidase and α-amylase Activities

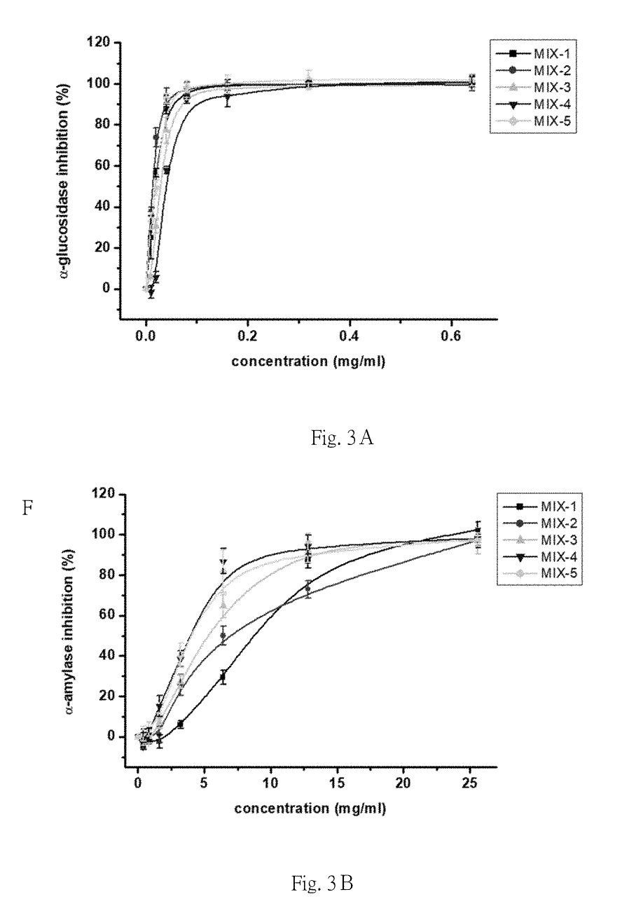

In the five natural aqueous extracts in different ratios, the result in FIG. 3 shows that the natural aqueous extract MIX-2 has the best α-glucosidase inhibition effect; the natural aqueous extracts MIX-4 and MIX-5 have better α-amylase inhibition effects; and the inhibition effects of natural aqueous extracts for α-glucosidase and α-amylase are better than the single use effect of C. osmophloeum or C. pinnatifida. It is inferred that the inhibition capability of the natural aqueous extract of C. pinnatifida and C. osmophloeum is improved under the interaction thereof. Therefore, the natural aqueous extract MIX-2 has the potential of being developed to be a blood glucose regulation product.

The effective concentration of the pharmaceutical composition in an enzyme inhibition cell experiment is 0.01 mg/ml-25.6 mg/ml. Taking a 65 kg weight adult as an example, the adult has about 5 L blood in the body, and the daily dose for a person is calculated to be 0.05 g-128 g, the preferred drug concentration is 0.055 mg/ml-6.4 mg/ml, and the preferred daily dose for a person is calculated to be 0.275 g-32 g, the optimal drug concentration is 0.055 mg/ml-0.32 mg/ml, and the optimal daily dose for a person is calculated to be 0.275 g-1.6 g.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A natural composition having an α-glucosidase activity inhibition effect, wherein the natural composition comprises C. osmophloeum and C. pinnatifida.

2. The natural composition as claimed in claim 1, wherein the C. osmophloeum is an aqueous extract of C. osmophloeum leaves.

3. The natural composition as claimed in claim 1, wherein the C. pinnatifida is an aqueous extract of C. pinnatifida fruit.

4. The natural composition as claimed in claim 1, wherein the weight ratio of the C. osmophloeum to the C. pinnatifida is 1:0.05-2.

5. The natural composition as claimed in claim 1, wherein the preferred weight ratio of the C. osmophloeum to the C. pinnatifida is 1:0.5-1.5.

6. The natural composition as claimed in claim 1, wherein the optimal weight ratio of the C. osmophloeum to the C. pinnatifida is 1:0.9-1.1.

7. The natural composition as claimed in claim 1, wherein the natural composition can further comprise a carrier, an excipient, a diluent and an adjuvant which are pharmaceutically acceptable.

8. A use of the natural composition as claimed in claims 1, wherein the drug can regulate blood glucose.

9. The use as claimed in claims 8, wherein the blood glucose is regulated by regulating the absorption of carbohydrate.

10. The use as claimed in claims 9, wherein the absorption of carbohydrate is regulated by inhibiting the activity of α-glucosidase.

11. The use as claimed in claim 8, wherein the drug can further inhibit the activity of α-amylase.

12. The use as claimed in claim 8, wherein the drug is administrated by means of oral administration, intravenous injection, or injection.

13. The use as claimed in claim 8, wherein the drug is administrated in the form of capsule, pastille, powder or oral liquid.

14. The use as claimed in claim 8, wherein the daily dose of the natural composition is 0.05-128 g.

15. The use as claimed in claim 8, wherein the preferred daily dose of the natural composition is 0.275-32 g.

16. The use as claimed in claim 8, wherein the optimal daily dose of the natural composition is 0.275-1.6 g.
